(19) United States
(12) Patent Application Publication
(10) Pub. No.: US 2007/0082017 A1
(43) Pub. Date: Apr. 12, 2007

(54) Lipid compositions and methods of use

(76) Inventor: Scheffer C.G. Tseng, Pinecrest, FL (US)

Correspondence Address: WILSON SONSINI GOODRICH & ROSATI, P.C.;ATTN: JEFFERY W. GUISE, ESQ, 12235 EL CAMINO REAL, SUITE 200, SAN DIEGO, CA, 92130-3002, US

(21) Appl. No.: 10/585,705

(22) PCT Filed: Jan. 9, 2005
(86) PCT No.: PCT/US05/00603

§ 371 Date: Jul. 8, 2006

Related U.S. Application Data
(60) Provisional application No. 60/535,597, filed on Jan. 10, 2004.

(51) Int. Cl. A61K 31/685; A61K 9/00

(52) U.S. Cl. 424/400; 514/78

(57) ABSTRACT

The present invention relates to a composition comprising: a C12 to C24 branched or unbranched hydrocarbon; a mid-chain triglyceride; a C26 to C36 branched or unbranched hydrocarbon; a cholesteryl ester; an ester of a C10 to C24 fatty acid and a C10 to C20 alcohol; an ester of a C10 to C24 fatty acid and a C21 to C34 alcohol; glycerol; and a polar lipid; and to methods of making and methods of using the composition to treat lipid tear deficiency (LTD), aqueous tear deficiency (ATD), a combination of LTD and ATD, and other dry eye conditions. The composition is substantially free of water and substantially free of an artificial surfactant. A method of administering an ointment comprising a lipid composition to treat dry eye, while achieving sustained release of the ointment and preventing a blurring of vision by the ointment, comprises applying the ointment to the inferior lid margin of the outside skin of the lower eyelid or to the superior lid margin of the outside skin of the upper eyelid, and allowing the ointment to diffuse onto the eye. Also disclosed is the use of kinetic analysis of tear interference images to analyze an individual's precorneal lipid film spread to identify LTD or to evaluate response to LTD treatment in order to adjust percentages of lipids in the composition.

### RELATED APPLICATION

This application claims the benefit of U.S. Provisional Application No. 60/535,597, filed on Jan. 10, 2004, the teachings of which are incorporated herein by reference in their entirety.

### BACKGROUND OF THE INVENTION

In the human eye, a stable precorneal tear film is essential for maintenance of a healthy, smooth, and comfortable ocular surface. Breakdown in the precorneal tear film can result in dehydration of the exposed outer surface of the eye, and symptoms of dryness such as a sensation of grittiness, irritation, burning, pain, redness, itching, blurred vision, and photophobia in mild cases, and in ulceration and infection in severe cases.

It is believed that the precorneal tear film is a complex fluid comprising three layers or phases, and that the absence of any one of the layer components causes discomfort and can lead to temporary or permanent dry eye syndromes. The inner layer immediately adjacent to the ocular surface is dominated by a thin layer of mucin about 0.02 microns thick. The mucin comprises a group of glycoproteins derived from goblet cells located in the conjunctiva or derived from corneal and conjunctival epithelial cells. The intermediate layer, about 7.0 microns thick, is an aqueous layer derived from the lacrimal gland and from the accessory lacrimal glands of Wolfring and Krause. The outermost layer, about 0.1 micron thick, is a layer of lipids derived primarily from the meibomian glands, also referred to as the tarsal gland, lining the upper and lower eye lid margins. In a healthy eye, the meibomian glands continuously produce meibum material comprising numerous types of lipids that are excreted onto the eyelid margin. In a normal healthy eye, the process of blinking spreads the lipids of the meibum material uniformly over the ocular surface to form the outer portion of the precorneal tear film. In addition, the tear film includes dispersed electrolytes and proteins.

Dry eye characterized by an unstable tear film can be generally categorized as “aqueous tear deficiency” (ATD); “lipid tear deficiency” (LTD); or a combination of both ATD and LTD. Although possible mechanisms in the pathogenesis of the condition referred to as “dry eye” continue to be a target of research, dry eye remains a common clinical problem. Currently available treatments for ATD include the frequent administration of various types of polymer-based artificial tears, preferably non-preserved, as tear substitutes. These artificial tears tend to yield only temporary relief. A typical polymer-based artificial tear may include dextran and hydroxypropyl methylcellulose polymer. Some preparations contain aqueous emulsions and a surfactant. Other treatments include punctal occlusion; administration of hormones such as androgens; and administration of cytokine-blocking agents such as cyclosporin A to suppress or interrupt the inflammatory response component of some dry eye disease processes. Yet another treatment is topical or oral administration of antibiotics, such as tetracycline. Unfortunately, there is as yet apparently no commercially available treatment for LTD.

To date, none of these treatments appear effective in most dry eye patients. Administration of existing tear substitutes needs to be repeated on a frequent basis, for example, from several times a day to hourly, depending on the severity of the dry eye condition. Thus, an on-going need exists for new and improved methods of differentiating the various dry eye states; a new and improved approach to restoring and maintaining the homeostasis of the tear film in a patient suffering from LTD, ATD, or a combination of LTD and ATD; and a new and improved composition and method of administration of the composition that achieves sustained release without unwanted side effects such as blurring of vision. To this point, such a composition and method of administration has not been identified or made available pharmaceutically for the treatment of a dry eye condition. There is a need for a therapeutic approach keyed to individual patient tear profiles or to patterns of tear spread on the corneal surface. There is also a need for a new and improved method of evaluating the clinical efficacy of a treatment for dry eye.

### SUMMARY OF THE INVENTION

It has now been found that compositions comprising: a C12 to C24 branched or unbranched hydrocarbon; a mid-chain triglyceride; a C26 to C36 branched or unbranched hydrocarbon; a cholesteryl ester; an ester of a C10 to C24 fatty acid and a C10 to C20 alcohol; an ester of a C10 to C24 fatty acid and a C21 to C34 alcohol; glycerol; and a polar lipid provide unexpectedly greater relief from the symptoms of dry eye than do other available preparations. Prolonged relief from the symptoms of dry eye caused by ATD, LTD, and a combination of both ATD and LTD is provided by exemplary compositions of the invention applied according to an embodiment of the method of the invention, for example, to the outside skin of the upper or the lower eyelid in an area adjacent to the lashes. An exemplary composition may be substantially free of water, and may also be substantially free of an artificial surfactant. The invention inter alia includes the following, alone or in combination.

One embodiment of the invention is a composition comprising: mineral oil or a mixture comprising C12 to C24 alkanes; a mid-chain triglyceride comprising a compound of the formula CH₂(OOCR₁)CH(OOCR₂)CH₂(OOCR)₃, wherein R₁, R₂, and R₃ are the same or different and are each independently a C6 to C12 branched or unbranched alkyl group; squalane; cholesteryl behenate; steraryl palmitate or palmitic acid steraryl ester; natural or artificial beeswax; glycerol; and L-α-phosphatidylcholine.

In another aspect, the invention relates to a method of making a composition for treatment of dry eyes in an individual in need thereof, the method comprising the steps of:
a) contacting mineral oil or a mixture comprising C12 to C24 alkanes; a mid-chain triglyceride comprising a compound of the formula CH₂(OOCR₁)CH(OOCR₂)CH₂(OOCR)₃, wherein R₁, R₂, and R₃ are the same or different and are each independently a C6 to C12 branched or unbranched alkyl group; a C26 to C36 branched or unbranched hydrocarbon; glycerol; and a polar lipid to produce a first mixture of ingredients;
b) maintaining the first mixture at first conditions sufficient to disperse the ingredients and form a first solution or a first suspension;
c) contacting the first mixture with a cholesteryl ester; an ester of a C10 to C24 fatty acid and a C10 to C20 alcohol; an ester of a C10 to C24 fatty acid and a C21 to C34 alcohol to produce a second mixture; and
d) maintaining the second mixture at second conditions sufficient to disperse the ingredients of the first mixture with the second mixture and thereby form the composition.

In another aspect, the invention relates to a method for treating a dry eye condition by administering an ointment comprising at least one lipid to an individual in need thereof, while achieving sustained release of the ointment and preventing a blurring of vision by the ointment, the method comprising administering a therapeutically effective amount of the ointment to the inferior lid margin of the outside skin of the lower eyelid or to the superior lid margin of the outside skin of the upper eyelid, and allowing the ointment to diffuse onto the eye surface.

Another embodiment of the invention is the use of a composition comprising a polar lipid and a non-polar lipid, wherein the composition is substantially free of water; substantially free of an artificial surfactant; and substantially free of an artificial polymer, in the manufacture of a medicament for the treatment of a condition chosen from LTD, ATD, a combination of LTD and ATD, epidermal dysplasia, Stevens Johnson Syndrome, meibomian gland diseases, rosacea, blepharitis, lagophthalmos, chemical injuries, thermal burn injuries, and diseases causing meibomian gland dysfunction.

Another embodiment of the invention is a method for treating dry eyes in an individual in need thereof, comprising:
a) using kinetic analysis of tear interference images to analyze a precorneal lipid film spread of the individual;
b) determining whether or not the precorneal lipid film spread is characteristic of LTD; and if the film spread is characteristic of LTD, administering a therapeutically effective amount of a composition comprising: a C12 to C24 branched or unbranched hydrocarbon; a mid-chain triglyceride; a C26 to C36 branched or unbranched hydrocarbon; a cholesteryl ester; an ester of a C10 to C24 fatty acid and a C10 to C20 alcohol; an ester of a C10 to C24 fatty acid and a C21 to C34 alcohol; glycerol; and a polar lipid.

Another embodiment of the invention is the use of the disclosed composition in the manufacture of a medicament for the treatment of a condition chosen from LTD, ATD, a combination of LTD and ATD, epidermal dysplasia, Stevens Johnson Syndrome, meibomian gland diseases, rosacea, blepharitis, lagophthalmos, chemical injuries, thermal burn injuries, and diseases causing meibomian gland dysfunction.

Another embodiment of the invention is the use of a composition comprising a polar lipid and a non-polar lipid, wherein the composition is substantially free of water; substantially free of an artificial surfactant; and substantially free of an artificial polymer, in the manufacture of a medicament for the treatment of a condition chosen from LTD, ATD, a combination of LTD and ATD, epidermal dysplasia, Stevens Johnson Syndrome, meibomian gland diseases, rosacea, blepharitis, lagophthalmos, chemical injuries, thermal burn injuries, and diseases causing meibomian gland dysfunction.

The lipid compositions of the invention are useful for restoring a stable, lipid tear film in the eye of an individual suffering from dry eye or from one or more conditions associated with dry eye. Another advantage for treatment of a dry eye condition provided by the present invention is an approach that may be keyed to individual patient tear profiles or to kinetic analysis of patterns of tear spread on the corneal surface. The composition may be varied depending on, for example, whether the dry eye condition is due to LTD, ATD, or a combination of LTD and ATD.

Further, in contrast to current methods for applying artificial tears directly to the ocular surface, the disclosed methods of delivery of disclosed lipid compositions allow the composition to diffuse onto the surface of the eye, thereby achieving sustained release of the composition: maximizing contact time of the composition with the cornea and conjunctiva; and preventing a blurring of vision by the composition, the blurring that would occur if the composition were placed excessively on the surface of the eye. In fact, the disclosed composition administered according to a disclosed method provides a film that not only lubricates the ocular surface and reduces the friction generated by lid blinking, but also improves the optical properties of the ocular surface of an eye with insufficient tear film production.

### BRIEF DESCRIPTION OF THE DRAWINGS

The foregoing and other features and advantages of the invention will be apparent from the following more particular description of illustrative embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a longitudinal sectional view of an ointment applicator (10) for use in applying the composition according to an embodiment of the invention.

FIG. 1A is a cross-sectional view of applicator (10) taken at the anterior end (44).

FIG. 2 is a longitudinal sectional view of housing (90) depicting guides (92) and assembly insertion of spindle (80).

FIG. 2A is an end view of applicator (10) taken at posterior end of spindle (80) at 2A-2A of FIG. 2 depicting periphery nodes (84).

FIG. 2B is a cross-sectional view of housing (90) taken at 2B-2B of FIG. 2 and depicting guides (92).

FIG. 2C is a cross-sectional view of housing (90) and spindle (80) assembly taken at 2C-2C of FIG. 2.

FIG. 3 depicts a partial sectional view of applicator (10) with housing (90) removed to more clearly show an assembly of spindle (80) having external spiral threads engaging two threaded tabs (46) connected to actuator (70).

FIG. 3A depicts assembly steps of FIG. 3.

### DETAILED DESCRIPTION OF THE INVENTION

The invention inter alia also includes the following exemplary embodiments, alone or in combination. It will be understood that the particular embodiments of the invention are shown by way of illustration and not as limitations of the invention. At the outset, the invention is described in its broadest overall aspects, with a more detailed description following. The features and other details of the compositions and methods of the invention will be further pointed out in the claims.

The present invention is directed to compositions and methods of treating a dry eye condition in an individual. The compositions include disclosed lipid compositions that may be, for example, in an ointment form. The term “methods of treating” when used in connection with the present invention means amelioration, prevention or relief from the symptoms and/or effects associated with a dry eye condition in an individual. The individual may be human or other mammal. The person of ordinary skill in the medical art recognizes that “prevention” of the symptoms and/or effects associated with dry eye is not an absolute term. In the medical art it is understood to refer to the prophylactic administration of a drug to substantially diminish the likelihood or seriousness of the condition.

The compositions of the invention are useful for treating dry eye. The dry eye condition may be LTD due to insufficient production of meibum. The disclosed compositions and disclosed methods for using the compositions can be used to restore the ocular surface to a condition approximating a condition wherein sufficient meibum is produced, thereby alleviating ocular irritation due to LTD. Furthermore, the disclosed compositions and methods can also be used to treat patients with ocular irritation caused by ATD, a combination of LTD and ATD, or a deficiency in the amount of mucin produced. Both ATD and mucin deficiency indirectly impair lipid tear film formation and stability.

Each of the component layers of the precorneal tear film, including the outer, lipid layer, the middle aqueous layer, and the inner mucin layer has a particular function. These components of tears are mechanically spread over the ocular surface through a neuronally-controlled lid blinking mechanism. Over a period of time, tears are cleared from the eye through the nasolacrimal drainage system into the nose, an action that is controlled by blinking of the eyelid. The absence of any one of the layer components causes discomfort and can lead to a temporary or a permanent dry eye syndrome. Disorders that disturb these compositional and hydrodynamic factors will invariably yield an unstable tear film (e.g., a preocular tear film that breaks up rapidly), a common hallmark of various dry eye states, and cause various symptoms of eye irritation, light sensitivity, fatigue, and pain.

The lipid layer (meibum): The outer, lipid layer of the tear film is expressed primarily from the meibomian gland, a specialized sebaceous gland on the upper and lower eyelids. The lipid layer of the tear film covers the aqueous layer and significantly retards evaporation of the underlying aqueous layer from the surface of the eye. Insufficient meibum production can lead to progressively increased water evaporation and thinning of the precorneal tear film, increased dryness of the corneal surface, the formation of dry spots on the cornea, and epithelial alteration of the cornea and the conjunctiva.

The lipid layer also functions to lower the surface tension of the tear film and increase the stability of the tear film. Another function of meibum lipids is to coat the lid margins and act as a barrier to prevent the development of chronic irritation along the skin of lids from constant wetting by aqueous tears. Yet another function of meibum lipids is to help lubricate the ocular surface during lid blinking to reduce blinking-related mechanical friction and decrease blinking mediated microtrauma. Phospholipid is a component of meibum, and may act as a natural surfactant forming an interface between the aqueous layer and the non-polar lipids of the lipid layer. (Cheol Hwa Song, et al., Enhanced Secretory Group II PLA2 Activity in the Tears of Chronic Blepharitis Patients, Investigative Ophthalmology and Visual Science. 40:2744-2748 (1999).)

When the meibum content in the tear film is decreased in conditions leading to LTD, these functions may not be adequately performed by the remaining meibum. Specifically, for example, the instability of the precorneal tear film is increased; that is, break-up time of the precorneal tear film is decreased. In addition, evaporation of the aqueous phase of the tear film is more rapid, and chronic irritation of the lid margins and skin occurs. The effects of decreased meibum may be observed in patients suffering from congenital ectodermal dysplasia, a rare abnormality associated with multiple developmental anomalies including partial or total absence of the meibomian glands. The resultant lack of meibum in the eyes of such patients causes an immediate break-up of the tear film, that results in severe changes of the ocular surface, including opacification of the cornea.

Another example of the lack of meibum is a patient suffering from different forms of meibomiam gland dysfunction. Chronic blepharitis is a fairly common condition among the elderly. This condition is characterized by diffuse inflammation around the meibomian gland orifices due to lipid secretions solidifying within the glands and squamous metaplasia of the meibomiam gland orifices, resulting in plugging of the orifices, with gland dilation, distortion, and atrophy. The lid margins become thickened and irregular, with dilated blood vessels. Tarsal injection with papillary hypertrophy, bulbar injection and superficial punctate keratopathy (SPK) frequently occurs. The latter is attributed to an unstable tear film that is seen clinically by rapid break-up time of the tear film. These changes in the tear film produce symptoms of burning, irritation, drying, grittiness, fatigue, and the like, as well as changes in visual acuity. When meibomian glands are not totally dysfunctional, fresh meibum from deep within the residual gland may be digitally expressed into the tear film, thereby slowing the break-up time. (McCulley J P, Sciallis G F, Meibomian keratoconjunctivitis, Am J. Ophthalmol. 84:788-793 (1996).)

The aqueous layer: This layer, the major component of tears, is secreted by lacrimal glands. The aqueous layer assists in providing oxygen to the cornea, and also comprises proteins, electrolytes, and water, other substances important to a healthy eye. ATD is more widely recognized than LTD. One form of ATD is keratoconjunctivitis sicca, an ocular surface disorder characterized by profound drying of the ocular surface and caused by diseased lacrimal glands.

The mucin layer (mucus) the mucin layer is a viscous material that lies on top of the epithelial cells of the cornea and under the aqueous layer of the tear film. In the absence of mucin, tears tend to bead up on the cornea. It is thus believed that mucin material assists in spread of the precorneal tear film and provides for interaction between the lipid layer and the aqueous layer. Mucin may be present throughout the entire fluid of the tear film, and not confined to the lower layer of the tear film.

Mucins are glycoproteins found in saliva, gastric juices, and the like that form viscous solutions and act as lubricants or protectants on external and internal body surfaces. Mucins are typically high molecular weight compounds, often greater than 100,000 daltons, and are extensively glycosylated (up to about 80% glycan). Mucins have been purified from bovine submaxillary glands, canine trachea, bovine gallbladder, rat submandibular salivary gland, and porcine stomach. Lipids can bind to the non-glycosylated protein domain of these mucins via hydrophobic interactions. This binding may modulate the interaction between polar phospholipids in the meibum and water in the aqueous layer. We believe that mucin in tears functions as a natural surfactant in tears, and that mucin concentration may be an important factor in determining the potency of an ophthalmic formulation in relation to mucin levels. Artificial surfactants, as required in some commercially available artificial tears, are not needed in the present invention, and are preferably excluded.

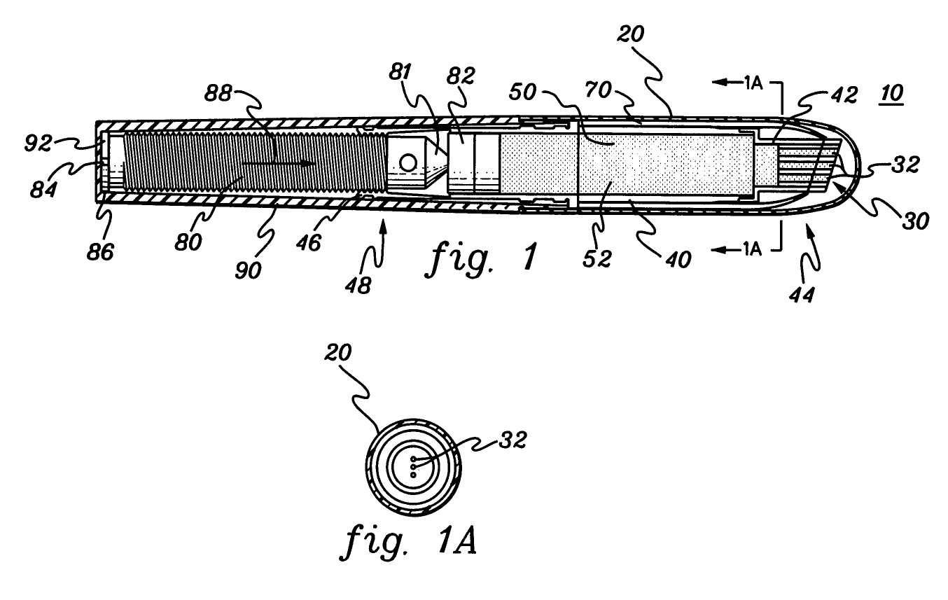

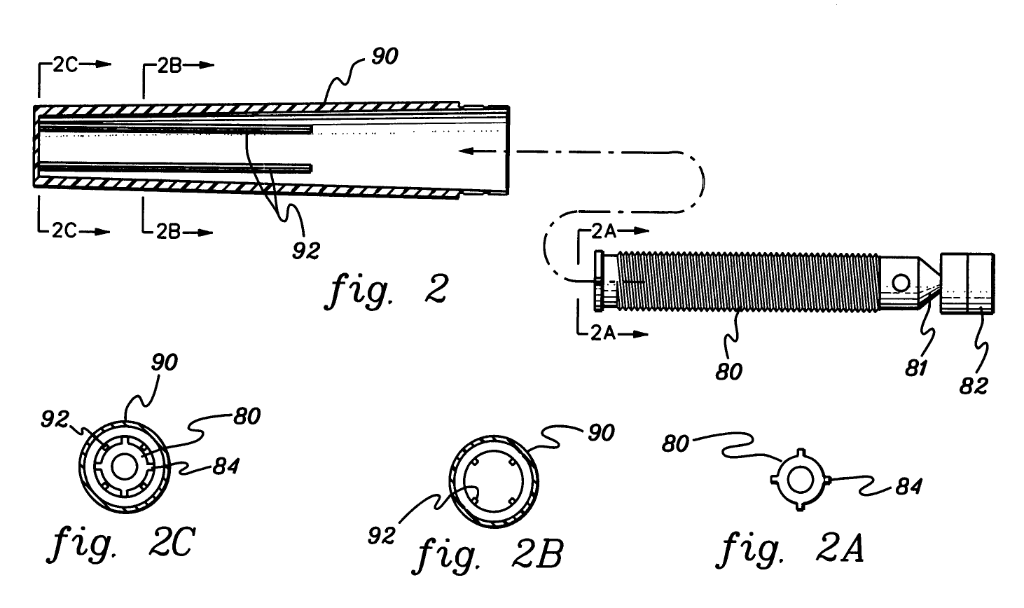

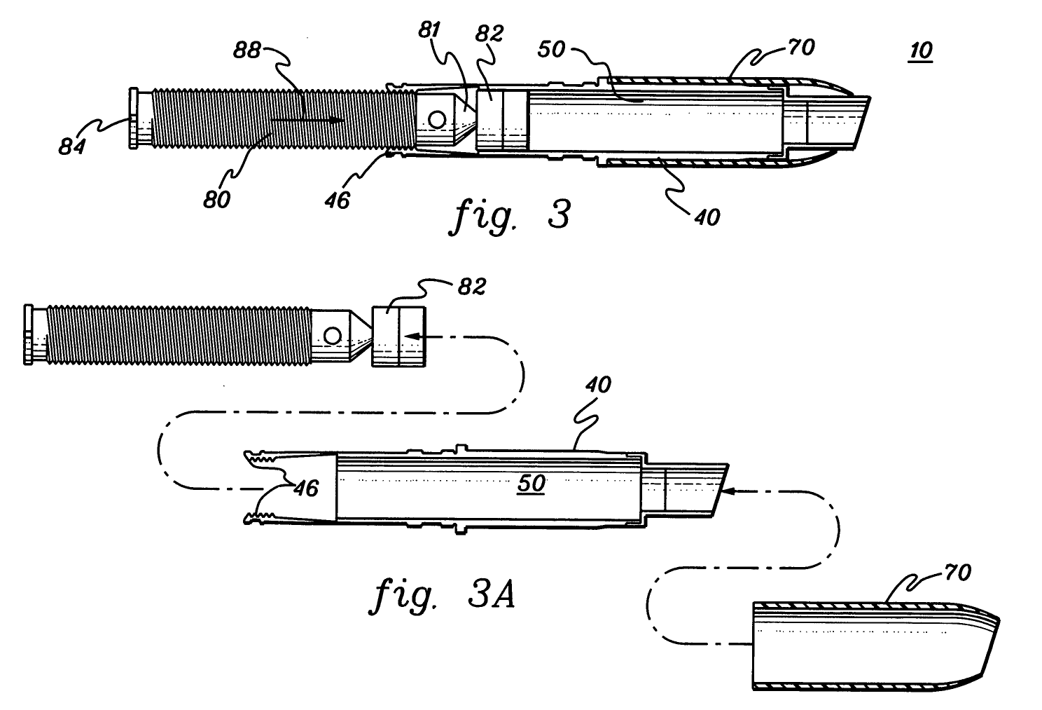

We have discovered in vitro that the concentration of mucin glycoprotein in tears affects lipid spread and thickness, and hence the stability of the tear film. If the mucin glycoprotein concentration in tears of a patient with dry eye syndrome is determined using an in vitro assay described herein, then the lipid content of a composition of the invention can be adjusted and optimized according to the mucin level. The stability of an abnormal tear film resulting from a deficiency of mucin components can be improved by applying an amount of an exemplary composition of the invention to the superior or inferior lid margin. In one embodiment of the invention, a disclosed lipid composition is applied to a lid margin, that is, the area of lid inside the eyelashes. It does not matter if the composition is brushed, rubbed, or smeared onto the skin outside the lashes. The composition may be applied to a lid margin with the use of an applicator similar to, for example, an applicator used to apply eyeliner or other eye makeup. An exemplary applicator is shown in FIG. 1 through FIG. 3A. An applicator may have one or several discharge openings, but in an exemplary embodiment, the entire diameter of the opening or openings should cover a width of from about one half (0.5) millimeter to about five (5) millimeters of eyelid in each application. The use of an applicator as disclosed permits controlled application of the disclosed composition to an area of eyelid skin just inside the lashes.

Stevens-Johnson syndrome can cause meibomian gland dysfunction and loss of goblet cells in the conjunctiva thereby resulting in the loss of the mucin layer and instability of the tear film. As a consequence, there may be undesirable changes in the ocular surface or even severe damage to the surface.

The diagnosis of ATD is straightforward, with the diagnostic criteria based on the measurement of aqueous tear production. The disclosed lipid compositions and disclosed methods of administration thereof can be used to increase the thickness of the lipid tear barrier, reducing the rate of aqueous tear evaporation and thereby treating even ATD as well as LTD.

Diagnosis of LTD is indirect, and it is important clinically to differentiate LTD from ATD. Current methods employed to diagnose LTD include, but are not limited to: examining the morphological change of meibomian gland using meibography (Robin J B, et al. In vivo transillumination biomicroscopy and photography of meibomian gland dysfunction; Ophthalmology; 92:1423-6 (1985).); and inferring by showing rapid tear evaporation or a combination of dye staining and impression cytology (Shimazaki J, et al. Meibomian gland dysfunction in patients with Sjögren syndrome; Ophthalmology; 105:1485-8 (1998).).

One non-invasive method of investigating the lipid tear layer of the precorneal tear film is by use of images of tear interference (TI). Kinetic analysis of TI images can be used to differentiate an LTD dry eye condition from an ATD dry eye. The teachings of U.S. patent application Ser. No. 10/131,665, filed on 24 Apr, 2002 by Tseng et al., for Apparatus and Method for the Kinetic Analysis of Tear Stability, published as US 2002/0180929 A1, are incorporated herein by reference in their entirety. The disclosed apparatus can be used to obtain a series of images illustrating a tear film and a lipid film dispersion pattern indicative of a tear stability condition, and can be used to determine the tear lipid layer thickness from look up tables. For an LTD patient, TI will produce a vertical pattern with slow spread time and low thickness, typical of LTD, but not ATD. Once the dry eye condition is diagnosed as LTD, the disclosed compositions can be administered according to a disclosed method of the invention.

Further, the composition administered to an LTD patient can be varied, with no more than routine experimentation, according to the diagnosis and the severity of the condition, without departing from the scope of the invention. Thus, kinetic analysis of tear interference according to the teachings of U.S. patent application Ser. No. 10/131,665 can be used to further refine the compositions of the present invention to better meet the needs of an individual patient and to evaluate the progress of the LTD patient who is being treated with the disclosed compositions and methods.

Utilizing the teachings of U.S. patent application Ser. No. 10/131,665, another embodiment of the invention is a method for treating dry eyes in an individual in need thereof, comprising:

using kinetic analysis of tear interference images to analyze a precorneal lipid film spread of the individual; determining whether or not the precorneal lipid film spread is characteristic of LTD or a combination of LTD and ATD; and if the film spread is characteristic of LTD or LTD and ATD, administering according to an embodiment of the method of the invention a therapeutically effective amount of a disclosed composition of the invention.

Lipids: Disclosed herein are compositions comprising a mixture of lipids of at least two different chemical structures, the mixtures being useful for restoring the stability of the lipid tear film in an individual in need thereof, and methods of administering the composition. As the term is used herein, lipids are a variety of organic molecules that include fatty acids, glycerides (glycerol-derived lipids), non-glyceride lipids including steroids, phospholipids, prostaglandins, terpenes, waxes, which are generally solid at room temperature, and complex lipids such as lipoproteins and glycolipids. Lipids are generally liquid at room temperature and are more soluble in nonpolar solvents than in polar solvents. Fatty acids are long, unbranched monocarboxylic acids containing from about 10 to about 24 carbon atoms. The pKa of a fatty acid is around 4.5. Therefore, generally fatty acids are neutral below pH 4.5 and are charged above pH 4.5. They typically have an even number of carbon atoms due to their biosynthetic pathway. Fatty acids are typically found as components of larger lipid species. The disclosed composition may comprise myricyl palmitate, for example.

Glycerides are lipid esters of the glycerol molecule, C₃H₅(OH)₃, and possess a three carbon “backbone” of glycerol. Esterification may occur at one, two or all three OH locations, producing monoglycerides, diglycerides, and triglycerides, respectively. The fatty acid groups can be the same or different and may be saturated or unsaturated. In one embodiment of the disclosed composition the triglycerides are mid-chain triglycerides comprising, for example, mixed mid-chain triglycerides (e.g., 6-12 carbons, or 8-10 carbons). The composition may comprise glycerides of caplorylic, capric, or fatty acids with longer carbon chain lengths; or mixtures thereof. Triglycerides are neutral lipids. A composition according to an embodiment of the invention comprises a mid-chain triglyceride of the formula CH₂(OOCR₁)CH(OOCR₂)CH₂(OOCR)₃, wherein R₁, R₂, and R₃ are the same or different and are each independently a C6 to C12 branched or unbranched alkyl group. “Alkyl”, as the term is used herein, is intended to include linear, branched, or cyclic hydrocarbon structures and combinations thereof.

Some of the compounds described herein may contain one or more asymmetric centers and may thus give rise to enantiomers, diastereomers, and other stereoisomeric forms that may be defined, in terms of absolute stereochemistry, as (R)— or (S)—. The present invention is meant to include all such possible isomers, as well as, their racemic and optically pure forms.

Phosphoglycerides: In contrast to triglycerides, phosphoglycerides, also referred to herein as phospholipids, are polar. Phospholipids are actually amphipathic (having both polar and nonpolar regions) molecules due to the presence of both a polar “head” and a nonpolar “tail” on the molecule. Phosphoglycerides have the glycerol backbone, two fatty acid residues or ester groups and a phosphoryl ester group bonded to the third alcohol carbon of the glycerol backbone. The simplest phosphoglyceride is phosphatidate. One embodiment of the disclosed composition comprises L-α-phosphatidyl choline, also known as lecithin, a phosphoglyceride made from the glycerol backbone, two fatty acids, and a phosphoryl ester wherein the R group of the ester is choline, HO—CH₂CH₂N(CH₃)₃⁺. A naturally occurring phospholipid, L-α-phosphatidyl choline is a major structural molecule found in brain tissue. An example of another phosphoglyceride that may be suitable for use in a composition according to an embodiment of the invention is L-α-phospatidyl ethanolamine, also referred to as cephalin. Similar to lecithin, cephalin is made from the glycerol backbone, two fatty acids, and a phosphoryl ester wherein the R group of the ester is ethanolamine, HO—CH₂CH₂NH₂. Commercially available cephalin may be isolated from sheep brain. Non-limiting examples of other phosphoglycerides that may be suitable for use in an embodiment of the invention include those wherein the R group of the phosphoryl ester is a polyol. Yet other examples of phosphoglycerides include lysophosphatidylcholine, phosphatidylserine, and cardiolipin. Non-limiting examples of fatty acid residues that may be bonded to the glycerol backbone of a phosphoglyceride include decanoate, dodecanoate, tetradecanoate, palmitate (hexadecanoate), stearate (octadecanoate), eicosanoate, cis-9-octadecnoate, cis, cis-9,12-octadecadienoate, and all cis-9,12,15-octadecatrienoate.

By applying a small amount of the formulation onto the superior or inferior lid margin, the lipid film can be replenished after each blink of the eyelid and remains stable over a long period of time. Therefore, this new treatment can be used to treat ocular irritation caused by abnormal lipid tear film resulting from an intrinsic deficiency of meibum lipids, or a deficiency of aqueous and/or mucin components.

A formulation according to an exemplary embodiment is preferably substantially free of water and does not contain an artificial surfactant. “Substantially free”, as the expression is used herein, means that water is not included in the formulation, but residual moisture may be present; for example, less than 1 percent (w/w) or 0.5 percent (w/w). The disclosed composition is preferably substantially free of water. Thus, the retention of lipids from the formulation is enhanced because the absence of water reduces drainage or outflow of the tears through the nasolacrimal system. Moreover, artificial surfactants are preferably excluded from the formulation of the invention because such artificial surfactants would interfere with lipid spread. “Artificial surfactants” as the term is used herein refers to non-naturally occurring surfactants such as polyoxyethylene fatty acid ethers and esters, and the other anionic, cationic, zwitterionic, and non-ionic surfactants listed below in Table 1.

TABLE 1

| | | |
|---|---|---|
| CMC | AGGREGATION | MOLECULAR |
| (mM) | NUMBER | WEIGHT |

Anionic

| | | | |
|---|---|---|---|
| Caprylic Acid, Sodium Salt | 351 | — | 166.2 |
| Cholic Acid, Sodium Salt | 14 | 2-4 | 430.6 |
| 1-Decanesulfonic Acid, Sodium Salt | 32.6 | — | 244.3 |
| Deoxycholic Acid, Sodium Salt | 5 | 4-10 | 414.6 |
| Glycocholic Acid, Sodium Salt | 7.1 | 2.1 | 487.6 |
| Glycodeoxycholic Acid, Sodium Salt | 2.1 | 2 | 471.6 |
| Lauryl Sulfate, Sodium Salt | 8.27 | 62 | 288.4 |
| Lauryl Sulfate, Lithium Salt | 7-10 | — | 272.3 |
| Taurocholic Acid, Sodium Salt | 3-11 | 4 | 537.7 |
| Taurodeoxycholic Acid, Sodium Salt | 1-4 | 6 | 521.7 |

Cationic

| | | | |
|---|---|---|---|
| Cetylpyridinium Chloride | 0.90 | — | 340.0 |
| Dodecyltrimethylammonium Bromide | 14 | — | 308.3 |
| Hexadecyltrimethylammonium Bromide | 1 | 169 | 364.5 |
| Tetradecyltrimethylammonium Bromide | 4-5 | 80 | 336.4 |

Zwitterionic

| | | | |
|---|---|---|---|
| N-Alkyl-N,N-dimethylammonio-1-propanesulfonates | | | |
| SB3-8 | — | — | 279.4 |
| SB3-10 | 25-40 | — | 307.5 |
| SB3-12 | 2-4 | 55 | 335.5 |
| SB3-14 | 0.1-0.4 | 83 | 363.6 |
| SB3-16 | 0.01-0.04 | — | 391.7 |
| SB3-18 | — | — | 419.7 |
| CHAPS | 6-10 | 4-14 | 614.9 |
| CHAPSO | 8 | 11 | 630.9 |

Non-Ionic

| | | | |
|---|---|---|---|
| BIGCHAP | 3-4 | 10 | 878.1 |
| Decanoyl-N-methylglucamide | 6-7 | — | 349.5 |
| Deoxy-BIGCHAP | 1.1-1.4 | 8-16 | 862.1 |
| n-Decyl β-D-glucopyranoside | 2-3 | — | 320.4 |
| n-Decyl β-D-maltoside | — | — | 510.6 |
| Digitonin | — | 60 | 1229.3 |
| n-Dodecyl β-D-glucopyranoside | 0.2 | — | 348.5 |
| n-Dodecyl β-D-maltoside | 0.1-0.6 | 98 | 510.6 |
| Heptanoyl-N-methylglucamide | — | — | 307.4 |
| n-Heptyl β-D-glucopyranoside | — | — | 278.4 |
| n-Heptyl β-D-thioglucopyranoside | 30 | — | 294.4 |
| n-Hexyl β-D-glucopyranoside | — | — | 264.3 |
| Nonanoyl-N-methylglucamide | 19-25 | — | 335.4 |
| Nonidet P-40 | 0.29 | — | 602.8 |
| n-Nonyl β-D-glucopyranoside | 6.5 | — | 306.4 |
| Octanoyl-N-methylglucamide | 58 | — | 321.4 |
| n-Octyl β-D-glucopyranoside | 20-25 | 84 | 292.4 |
| n-Octyl α-D-glucopyranoside | 10 | — | 292.4 |
| n-Octyl β-D-thioglucopyranoside | 9 | — | 308.4 |
| Pluronic F-68 | 0.04 | — | 8350 |
| Polyoxyethylene 23 lauryl ether (Brij 35) | 0.05-0.1 | 20-40 | 1225 |
| Polyoxyethylene sorbitan monolaurate (Tween 20) | 0.06 | — | 1228 |
| Polyoxyethylene sorbitan monooleate (Tween 80) | 0.012 | 60 | 1310 |
| Saponin | — | — | varies |
| n-Tetradecyl β-D-maltoside | — | — | 538.8 |
| Triton X-100 | 0.24 | 140 | 625 |
| Triton X-114 | 0.2 | — | 537 |
| n-Undecyl β-D-glucopyranoside | — | — | 334.5 |

An exemplary embodiment of a lipid composition according to the invention is disclosed below in Table 2. Table 2 and other examples are meant to be illustrative of the present invention; however, the practice of the invention is not limited or restricted in any way by it. The composition described in Table 2 has been designated “Composition A”. When used by individuals without ophthalmic disease and by some patients, Composition A stabilized the precorneal tear film with a prolonged effect when applied onto the lid margin. For example, Composition A, exemplified below is effective for a period of at least 12 to 24 hours after one application to the lid margin.

TABLE 2
Components of Lipid Composition A

| | Component | g/batch | percent (w/w) |
|---|---|---|---|
| 1 | Mineral oil | 13.55 | 52.7 |
| 2 | Mid-chain triglycerides (MCT) | 1.33 | 5.2 |
| 3 | Squalane | 4.68 | 18.2 |
| 4 | Cholesteryl behenate | 2.29 | 8.9 |
| 5 | Palmitic acid steraryl ester | 1.13 | 4.4 |
| 6 | Beeswax | 1.15 | 4.5 |
| 7 | Glycerol | 0.34 | 1.3 |
| 8 | L-α-phosphatidylcholine | 1.26 | 4.9 |

The sources of components used in preparing lipid Composition A are as follows:

| Name of | Name of | | |
|---|---|---|---|
| Company | Chemical | Catalog # | Lot # |
| E. R. Squibb | Mineral oil | 003-0559-52 9 | E 27870 |
| & Son, Inc. | | | |
| Mead Johnson | Mid-chain | 0087-0365-03 | P5448 |
| & Co. | triglycerides | | |
| | (MCT) | | |
| Sigma | Squalane | S-4510 | 15H2510 |
| Chemical Co. | | | |
| Sigma | Cholesteryl | C-6509 | 97F0955 |
| Chemical Co. | behenate | | |
| Sigma | Palmitate | P-3512 | 115H0981 |
| Chemical Co. | stearyl ester | | |
| Sigma | Glycerol (AR) | G-7757 | 53H0629 |
| Chemical Co. | | | |
| Aldrich | Beeswax | 24322-1 | 07623PG |
| Chemical | | | |
| American | 93.7% L-α- | | |
| Lecithin Co. | phosphatidylcholine | | |
| | (PHOSPHOLIPON 90) | | |

L-α-phosphatidylcholine may also be obtained in greater than 95% purity from American Lecithin (PHOSPHOLIPON 100 G-No. 110561). Lipid “Composition B” was made by using mineral oil, mid-chain triglycerides, squalane, cholesterol behenate, palmitic acid steraryl ester, beeswax, and glycerol as described above and greater than 95% pure L-α-phosphatidylcholine (PHOSPHOLIPON 100 G) in the amounts disclosed in Table 2. Both Compositions A and B were used by our patients with dry eye symptoms and provided extended relief from symptoms for a period of 12 to 24 hours after each application of the composition to the eyelid margin. This in vivo study is described below in the Clinical Trials section.

Method of Making a Lipid Composition:

Another embodiment of the invention is a method of making the lipid composition, also referred to herein as the “lipid ointment”, to be used in the treatment of a dry eye condition. In general, the compositions of the present invention may be prepared by the method described below, or by modifications thereof, using readily available starting materials, reagents and conventional synthesis procedures. In this method, it is also possible to make use of variants that are in themselves known, but are not mentioned here. One skilled in the art will recognize that the method of making a lipid composition according to one embodiment of the invention can be adapted to prepare other compositions of the invention. Following is a summary of the preparation of the disclosed lipid ointment, Composition A.

All equipment was sterilized prior to preparation of the lipid ointment. The entire process of measuring ingredients and compounding the lipid ointment was conducted in the laminar flow hood by an individual wearing surgical cap, mask, and gloves. While measuring ingredients, the flow of the laminar hood was stopped to avoid error. Before use, the L-α-lecithin was kept refrigerated at about 4 degrees Celsius (4° C.). Using forceps and scissors, small fragments of the L-α-lecithin were made on a sterile weigh boat. Under a sterile laminar flow hood, the following ingredients were added, in the order given, to a sterile 50 ml tube:

| | | | |
|---|---|---|---|
| Mineral oil (light) | 13.55 | grams | (Spectrum Chemical no. |
| | | | M 1501) |
| Medium-chain | 1.328 | grams | (Mead Johnson no. |
| triglycerides | | | 0056.64) |
| Glycerol | 0.338 | gram | (Sigma Chemical no. |
| | | | G7893) |
| Squalane | 4.68 | grams | (Sigma Chemical no. |
| | | | S4510) |
| L-α-lecithin | 1.26 | grams | (American Lecithin Co. |
| (Phospholipon 100G) | | | no. 110561) |

After the above five ingredients were added to the tube, the tube was warmed in an 80° C. water bath for one to two hours, with agitation by vortexing about every 5 minutes until the L-α-phosphatidylcholine was completely melted and all the ingredients were completely dissolved or dispersed to form a first mixture, which had a homogeneous appearance. The first mixture was cloudy. The following ingredients were then added to the first mixture in the tube, in the order given:

| Cholesterol | 2.295 grams | (Sigma Chemical no. C-6509) |
|---|---|---|
| behenate | | |
| Palmitic acid | 1.125 grams | (Sigma Chemical no. P-3512) |
| steraryl ester | | |
| Beeswax | 1.148 grams | (Aldrich Chemical Co. no. |
| | | 24,322-1) |

After the addition, the last three ingredients were mixed together with the first mixture to produce a second mixture by placing the tube in a water bath at a temperature of about 80° C. for about 15 to 30 minutes, vortexing about every 5 minutes during warming in the water bath until all the ingredients are dispersed and the second mixture had a homogeneous appearance, thereby forming the lipid composition. It should be noted that vortexing is needed to achieve solubilization of the solids with the first four ingredients, which are liquid at room temperature. It is important to maintain the first mixture and the second mixture at a temperature of from about 50° C. to about 95° C., preferably at about 80° C. The water bath was brought near the hood to keep the composition at about 80° C. at all times when the dispensing tubes or containers are being filled with the composition. Vortexing was also done when the tubes were removed from the water bath; and the lipid mixture was poured into the sterile ointment tubes quickly in order to prevent solidification. Care was taken to avoid water or alcohol contamination of the composition as it was packed into the ointment tubes. After cooling down to the room temperature, the ointment formed was used in lipid replacement therapy.

Variations on the above formulation are within the scope of the invention. Preferably, different formulations may be prepared for patients by altering the lipid composition according to the type of dry eye condition, the severity of the condition, an analysis of images of a patient's tear interference pattern, or the concentration of mucin glycoprotein in tears from a patient in need of treatment.

The disclosed formulation may comprise branched or unbranched, saturated or unsaturated hydrocarbons including mineral oil, petrolatum, squalane, and squalene. “Hydrocarbon” includes alkyl, cycloalkyl, alkenyl, alkynl, aryl, and combinations thereof. Mineral oil and petrolatum are mixtures of primarily linear hydrocarbons, but may include a small percentage of cyclic hydrocarbons. In an exemplary embodiment, mineral oil or a mixture of primarily C12 to C24 hydrocarbons may be present from about 10 percent (w/w) to about 65 percent (w/w) of the formulation, or from about 35 percent to about 65 percent of the formulation or from about 40 percent to about 60 percent of the formulation (or simply the remainder of the formulation). One embodiment of the lipid composition comprises a C26 to C36 hydrocarbon, for example, squalane (C₃₀H₆₂) or squalene (C₃₀H₅₀), present from about 5 percent (w/w) to about 30 percent (w/w), or from about 10 percent to about 25 percent.

The disclosed formulation may comprise a mixture of at least two different lipids which may include monoglycerides, diglycerides, triglycerides, free cholesterol, cholesterol esters, fatty acid esters, wax esters, glycols, polar lipid, free fatty acids, fatty alcohols, or the like. The concentration of each lipid component may be from about 0.5 percent to about 60 percent. See U.S. Pat. Nos. 4,866,049 and 5,278,151, the teachings of which are incorporated herein by reference, for a non-limiting list of examples of lipid sources. Non-limiting examples of fatty acid esters include myristyl palmitate and myricyl palmitate.

Monoglycerides are optionally present from about 1 percent (w/w) to about 10 percent (w/w). Diglycerides are optionally present from about 1 percent (w/w) to about 10 percent (w/w). Triglycerides may be, for example, mixed mid-chain triglycerides (e.g., 6-12 carbons, or 8-10 carbons); glycerides of caplorylic, capric, or fatty acids with longer carbon chain lengths; or mixtures thereof. Such triglycerides may be present from about 1 percent (w/w) to about 20 percent (w/w); from about 2 percent to about 20 percent; from about 1 percent to about 10 percent; or from about 1 percent to about 15 percent.

Examples of cholesteryl esters suitable for use in an embodiment include, but are not limited to, cholesteryl arachidate, cholesteryl behenate, cholesteryl palmitate, and cholesteryl oleate. In an exemplary embodiment, cholesteryl esters are present from about 2 percent (w/w) to about 35 percent (w/w); from about 5 percent to about 35 percent; or from about 5 percent to about 15 percent.

Fatty acid esters and wax esters suitable for use in an embodiment include, for example, but are not limited to: palmitic acid steraryl ester, beeswax, artificial beeswax, palmitic acid arachidyl ester, palmitoleic acid steraryl ester, or mixtures thereof. The fatty acid esters may be esters of a C10 to C24 fatty acid and a C10 to C20 alcohol or a C21 to C34 alcohol, for example. The disclosed composition may comprise about 2 percent (w/w) to about 35 percent (w/w) or about 2 percent to about 15 percent fatty acid esters and wax esters.

Beeswax is the major component of honeycomb and is produced by bees. The main component of beeswax is myricyl palmitate of formula C₃₀H₆₁—COO—C₁₅H₃₁. Beeswax also may comprise free cerotic acid, CH₃(CH₂)₁₄COOH, also refered to as hexacosanoic acid, triancontanol of formula CH₃(CH₂)₂₉OH, an ester of cerotic acid and triancontanol, long-chain alkanes such as hentriacontane of formula CH₃(CH₂)₂₉CH₃, polyesters, and hydroxyesters. The beeswax suitable for use in a disclosed composition may be bleached or unbleached. Artificial beeswax may also be used in a disclosed composition. Artificial or synthetic beeswax is the reaction product of blended organic acids and blended alcohols that simulate the composition of natural beeswax. Artificial beeswax primarily comprises alkyl esters of C16 to C32 fatty acids and C22 to C34 alcohols.

In a composition according to an embodiment of the invention, glycols of various carbon chain lengths (e.g., glycerol) may be present from about 0.5 percent (w/w) to about 5 percent (w/w). Optionally, free cholesterol, plant sterols, or mixtures thereof are present from about 1 percent (w/w) to about 5 percent (w/w). Free fatty acids such as, for example, palmitic acid, palmitoleic acid, oleic acid, linoleic acid, or mixtures thereof are optionally present from about 1 percent (w/w) to about 5 percent (w/w).

The disclosed composition may comprise polar lipids such as, for example, a phospholipid (e.g., sphingomyelin, phosphatidic acid, 1-α-phosphatidylcholine, phosphatidylinositol, phosphatidylserine, phosphatidylethanolamine, phosphatidylglycerol). Such polar lipids may be present from about 0.5 percent (w/w) to about 20 percent (w/w), or from about 1 percent to about 10 percent.

As previously described, only negligible amounts of water would be present in our formulation and artificial surfactants are preferentially excluded from our formulation. The disclosed composition does not include an aqueous solution, eye drops, or liposomes, none of which, based on our clinical experience, form a stable lipid film in a patient's eye. Such formulations would rapidly clear from the eye surface and therefore would not achieve a prolonged therapeutic effect (i.e., lasting over 24 hours in some patients) as observed in embodiments of the present invention. The disclosed compositions are ophthalmic formulations designed and tested according to kinetic analysis of tear film spread and stability in dry eye and normal individuals. The analysis of tear film spread and stability was based on images of tear interference patterns and on in vitro studies of lipid mixtures including varying concentrations of mucins. The rate of spread, thickness, and stability of the lipid film are dependent in part on the concentration of mucin in tears. The latter situation can result from aqueous and/or mucin tear deficiency in a patient with dry eye. A formulation and a method of treatment are provided by the present invention to supplement the ocular surface with a mixture of lipid.

One embodiment of the invention is a composition comprising: from about 35 percent to about 65 percent by weight of mineral oil or a mixture comprising C12 to C24 alkanes; from about 1 percent to about 15 percent by weight of a mid-chain triglyceride comprising a compound of the formula CH₂(OOCR₁)CH(OOCR₂)CH₂(OOCR)₃, wherein R₁, R₂, and R₃ are the same or different and are each independently a C6 to C12 branched or unbranched alkyl group; from about 10 percent to about 25 percent by weight of squalane; from about 5 percent to about 15 percent by weight of cholesteryl behenate; from about 2 percent to about 15 percent by weight of steraryl palmitate or palmitic acid steraryl ester; from about 2 percent to about 15 percent by weight of the ester of natural or artificial beeswax; from about 0.5 percent to about 5 percent by weight of glycerol; and from about 2 percent to about 10 percent by weight of the L-α-phosphatidylcholine. In one exemplary embodiment of the composition the mineral oil or the mixture comprising C12 to C24 alkanes may be from about 40 percent to about 60 percent by weight of the composition, and the mid-chain triglyceride may be from about 1 percent to about 10 percent by weight of the composition.

In addition to the above ingredients, there may also be incorporated other pharmaceutically acceptable additives including, for example, buffers, diluents, binders, stabilizers, and preservatives. A neutral pH is preferred in the range of between about 6.0 and about 7.8, more preferably between about 6.5 and about 7.4. Preferably, non-naturally occurring or artificial surfactants (e.g., classified as anionic, cationic, zwitterionic, non-ionic, see Table 1) are not included in the formulation. Preferably, water is not included.

Preferably the formulation is formed as an ointment, cream, or paste, but other forms known in the art may be used. Compounds for ophthalmic use may be formulated as described in Remington's Pharmaceutical Sciences (Maack Publishing Co., Easton, Pa.), the teachings of which are incorporated herein by reference. The relative amounts of effective ingredients in the compositions of the invention can be adjusted appropriately for efficacious administration to a patient, depending on the patient's condition.

Prior to testing Compositions A and B in vivo, the compositions were tested in an in vitro assay developed by the inventor. Several other different formulations were prepared prior to discovering the Compositions A and B. These compositions, including Compositions I to IV, disclosed in Table 3 below, were also tested in an in vitro assay, and in vivo for lipid replacement therapy.

TABLE 3
Other Lipid Compositions (I to IV)

| | | I | II | III | IV |
|---|---|---|---|---|---|
| | Component | (w/w %) | (w/w %) | (w/w %) | w/w %) |
| 1 | Mineral oil | 43.3 | 28.2 | 63.2 | 43.0 |
| 2 | Mid-chain | 8.7 | 4.1 | 4.0 | 6.3 |
| | triglycerides | | | | |
| 3 | Squalane | 0.0 | 48.4 | 14.2 | 22.3 |
| 4 | Cholesteryl behenate | 22.3 | 7.2 | 6.9 | 10.9 |
| 5 | Palmitic acid | 5.7 | 3.5 | 3.4 | 3.5 |
| | steraryl ester | | | | |
| 6 | Beeswax | 5.8 | 3.6 | 3.5 | 5.5 |
| 7 | Glycerol | 3.5 | 1.1 | 1.0 | 2.5 |
| 8 | L-α- | 10.7 | 3.9 | 3.8 | 6.0 |
| | phosphatidylcholine | | | | |

Only lipid mixtures A and B formed a desired lipid pattern in the in vitro assay. That is, lipid mixtures I, II, III, and IV did not form the desired lipid pattern. The formulation described in Table 2 yielded a stable lipid film based on an in vitro assay system we developed for the mucin concentrations from about 0.16 microgram/milliliter (μg/ml) to about 8 μg/ml. Only Compositions A and B, administered according to an embodiment of the method of the invention, proved to be highly effective in treating dry eye conditions in in vivo testing. Lipid compositions I, II, III, and IV did not prove to be highly effective in treating dry eye conditions in vivo.

Method of Treatment and Method of Administration of the Composition:

To achieve the improvements described herein, the present invention provides for a route of administration that allows for slow release of the lipids and other ingredients of the disclosed composition, thereby preventing unwanted side-effects such as blurring of vision by the ointment, and thereby reducing the number of administrations necessary to provide extended relief, compared to other possible methods of applying an ointment.

An embodiment of the invention is a method for treating a disorder chosen from LTD; ATD; a combination of LTD and ATD; epidermal dysplasia; Stevens Johnson Syndrome; meibomian gland diseases; rosacea; blepharitis; lagophthalmos; chemical injuries; thermal burn injuries; and diseases causing meibomian gland dysfunction, comprising administering to an individual in need thereof a therapeutically effective amount of the composition.

As used herein, the term “therapeutically effective amount” and grammatical variations thereof, refers to the amount of the composition or active agent required to be administered in order to induce a desired result in a patient. The result may be the alleviation (complete or partial) of the symptoms of dry eye. Typically, the composition is administered for a sufficient period of time to achieve the desired effect. Therapeutic efficacy may be determined as described herein and by using standard pharmacological procedures in experimental animals or humans.

A method for treating a dry eye condition in an individual in need thereof includes administering a therapeutically effective amount of the disclosed composition. In one embodiment, the composition is administered by a method comprising applying the composition to the outside skin of a lower eyelid or to the outside skin of an upper eyelid, and allowing the composition to diffuse onto the eye surface, thereby achieving sustained release of the composition and preventing or minimizing blurring of vision by the composition. For example, in an exemplary embodiment, the composition is applied to the inferior lid margin of the lower eyelid or to the superior lid margin of the upper eyelid. Additionally, a pharmaceutically active substance chosen from a steroid, an antibiotic, lipocalin, lactoferrin, lysozyme, cytokine-blocking agents such as cyclosporin A, and an antioxidant may be administered simultaneously, separately, or sequentially; and topically to the skin, to the ocular surface, or orally, to the patient in need thereof. TH2 inhibitors, FK-506, GATA3, and anti-T cell agents CD4, CD23, and systemic tetracycline may also be administered simultaneously, separately, or sequentially.

An effective amount of the disclosed composition needed to treat dry eye may depend upon a number of factors including, for example, the age and general health of the patient, the precise condition (e.g., deficiency of meibum lipids or a deficiency of aqueous and/or mucin tear components in the patient's tears) requiring treatment and its severity, and the patient's level of physical activity. The precise amount, number of doses, and timing of doses will ultimately be at the discretion of the attending physician, but preferably application is once or twice a day. For example, about 5 micrograms to about 10 micrograms of the formulation may be applied to each lid margin of a patient's eye; after blinking, this amount of the lipid mixture is sufficient to form a lipid film stable for about 12 to 24 hours.

Further embodiments of the invention include methods of administering the formulation to a patient to treat LTD, ATD, or a combination of LTD and ATD. In general, the formulation is applied onto the superior or inferior lid margin to provide prolonged relief of dry eye. In an exemplary embodiment of the method of the invention, application of the disclosed formulation is made to the superior or inferior lid margin, the site proximate to the site where the meibomian gland secretes its contents. This allows small quantities of the formulation to diffuse toward the ocular surface, contact fluid in the tear meniscus, and enter the preocular tear film. The formulation may be applied near the base of the eyelashes, manually, by cotton applicator, or with a brush such as that used to apply eyeliner.

The disclosed composition may be applied to the outside skin of the lower eyelid and to the outside skin of the upper eyelid at least once a day to about 6 times per day for a period of time sufficient to obtain an improvement in the dry eye condition or a decrease in severity of a symptom of the dry eye condition. For example, the composition may be applied to about one square centimeter of eyelid surface in each administration.

The composition may be applied from a tube or from a syringe-type applicator wherein the composition is applied by urging the ointment out of at least one discharge aperture in the syringe. One embodiment of the method of the invention is carried out by use of an ointment applicator (10), the design of which is shown in FIGS. 1 through 3A. The ointment applicator (10) can be used to controllably apply the disclosed composition close to the upper or lower lid margins.

FIG. 1 depicts a longitudinal sectional view of an ointment applicator (10) for use in one exemplary embodiment of the method of the invention. In one embodiment, the applicator (10) comprises a soft applicator tip (30) protected by a removable cap (20). The cap (20) is frictionally fit over a rotatable actuator (70) that is in turn frictionally fit over a hollow housing (40) that defines a wall of a reservoir (50) that holds ointment (52). A soft applicator tip (30) is inserted into an aperture (42) in the anterior end (44). Two threaded tabs (46) are connected to the actuator (70) and also contact a cylindrical-shaped spindle (80) positioned within a housing (90) of the applicator (10). The threaded tabs (46) are positioned to contact the spindle (80) at a location (48) posterior to the leading edge (81) of the spindle. The outer surface of spindle (80) comprises external spiral threads capable of frictionally engaging tabs (46).

The external spiral-threaded spindle (80), ensconced in housing (90), comprises a conical leading edge (81) in contact with, or attached to, a rotatable plunger (82). The spindle (80) also comprises nodes (84) on its posterior periphery (86). In the embodiment shown in FIG. 1, there are four nodes (84). Housing (90), in the embodiment shown, has four internal guides (92) positioned to engage the nodes (84) of spindle (80) to prevent spindle 80 from rotating beyond the impingement of nodes (84) and guides (92), and to direct the forward lateral travel (88) of spindle (80). The nodes (84) and guides (92) prevent spindle (80) from rotating through more than 90 degrees or one quarter of a rotation. Arrow (88) shows the direction of lateral travel of spindle (80) and plunger (82) as plunger (82) advances into reservoir (50).

By rotating actuator (70) in a clockwise direction, tabs (46) engage the external spiral threads of spindle (80), thereby pulling spindle (80) and plunger (82) forward and advancing plunger (82) into the reservoir (50), forcing ointment (52) out through the restricted discharge apertures (32) in applicator tip (30). The applicator shown in FIG. 1 is a modification of a commercially available applicator used for lipstick. One of the modifications is the number of apertures (32) and the diameter of each aperture (32). In an exemplary embodiment, there are from about one to about five apertures, each having a diameter of from about 0.1 millimeter to about 5 millimeters. The number of apertures and the diameter of each determine the area of skin that will be covered with ointment in each application or pass of the applicator over the skin. The actuator is rotated by hand to allow ointment to be released from the reservoir in a controlled fashion as the applicator is brushed over the skin of the upper and the lower eyelids as close as possible to the base of the eye lashes. Use of an applicator similar to an embodiment of the ointment applicator (10) depicted in FIG. 1 allows for a more controlled release of the ointment than would be obtained with a squeeze tube or with a simple plunger or syringe type applicator. After application of the composition to the lid margin via applicator, additional smearing of the composition by the fingertip can also be used.

To use applicator (10) to apply a narrow strip of ointment to the eyelid, the anterior aperture (42) of the applicator (10) is placed on the skin of the eyelid close to the lid margin; the rotating actuator (70) is rotated to cause tabs (46) to engage the external threads of spindle (80) and pull the plunger (82) in contact with the spindle (80) from the first position to the second position, thereby controllably urging a portion of composition (52) through the aperture (32) for application to the skin of the eyelid.

Summary of Initial In Vitro Assays:

An enzyme-linked immunosorbance assay (ELISA) based on a lectin from Helix promatia (HPA) and an antibody to mucosal epithelial mucin (MEM) shows that the mucin concentration in the tears of a group of normal volunteers ranged from an equivalent of 0.21 microgram per milliliter (μg/ml) to 0.25 μg/ml porcine stomach mucin (PSM). That is, the ELISA was calibrated with a standard amount of PSM. Older patients at risk for developing dry eye had an equivalent of about 0.75 μg/ml PSM (average).

An amount of porcine stomach mucin (PSM) believed to be equivalent to the amount of mucin present in a patient's tears was used in the initial in vitro assays designed to test the experimental formulations. A summary of some of these assays follows.

Purified mucin (Sigma) was filtered and dissolved in BSS ophthalmic irrigating solution (Alcon Laboratories), filtered via a 0.45 mm pore size to remove any insoluble debris, and serially diluted. For each assay, 5 ml of BSS solution with or without mucin was pre-warmed to 32° C. to mimic the corneal surface temperature, and added to a 35-mm petri dish (Becton Dickinson). A plastic ring about the height of the dish was precut from a 3.5 ml, plastic transfer pipette and placed in the center of dish. A droplet with an average of 0.14 μg of the lipid mixture was applied via a stainless spatula onto the solution surface in the center of the ring. The lipid composition spread with a characteristic pattern and its final appearance was visualized via a TEARSCOPE light source (Keeler Instruments) and imaged with a three-chip color CCD camera (PANASONIC® mounted on a dissecting stereomicroscope with 1× to 2× magnification (Carl Zeiss). The TEARSCOPE light source, an instrument that measures the thinness of the lipid layer based on the interference pattern, provides even illumination from all directions in the hemisphere above the dish.

For each tested condition, frames of serial video images were sampled uniformly (e.g., with 20 sec spacing in between) over time from the time of application (zero) to 100 sec and digitized using a METEOR frame grabber (MATROX® Electronics System). The percentage of area covered by the lipid film was calculated.

Results of In Vitro Testing:

Several different formulations of lipid mixtures that could spontaneously spread into a thin film upon application to a BSS solution surface at room temperature were assayed. The four lipid mixtures I, II, III and IV did not form a stable film in this in vitro assay. However, when the complete lipid Composition A was assayed, a stable thin film was rapidly formed. Application of an average of 0.14 μg of lipid Composition A, was sufficient to cover an area of about 0.75 cm².

When purified and filtered mucin was added to the BSS solution at a concentration of 0.16 μg/ml, addition of the lipid Composition A, resulted in a different lipid film, that was clearly visible under the TEARSCOPE light source. The film, grayish in appearance and lacking a color fringe, included numerous small round areas covered with a thinner film appearing as dark areas, and some aggregated granules. When the mucin concentration was raised to 0.8 μg/ml, a similar film was formed except that it had fewer aggregated granules; it had a smooth border; and the area covered by the thinner film was smaller than that mentioned above. Such a pattern mimics the condition seen in normal eyes and is a desired lipid pattern achieved by the formulations of the present invention.

When the mucin concentration was increased to 8 μg/ml, the area covered by the lipid film was similar to that of 0.8 μg/ml mucin but contained more insoluble granules. To estimate the speed of lipid spread, the border of each resultant film of selected frames was digitized and the lipid-covered area was calculated. Without mucin, the thin film rapidly expanded to the plastic ring. When mucin was added at a concentration of 0.16 μg/ml, lipid spread was also rapid and reached the border limited by the plastic ring. Analysis showed that spread of the lipid film was retarded by increased mucin concentrations.

Although the speed was progressively slower, the lipid mixture comprising mucin at concentrations of 0.16, 0.8, and 8 μg/ml, respectively, still could reach the ring border. However, at higher concentrations than 8 μg/ml, the lipid film was too small to achieve this. Further analysis of results confirmed that indeed increased mucin concentrations retarded lipid spread.

In this in vitro study, we observed that interactions between a test lipid mixture, Composition A, and mucin-containing balanced salt solutions affect spontaneous lipid spread. In particular, the speed of such spread was retarded by increasing concentrations of mucin present in BSS solution, resulting in increased film thickness and a different appearance. This finding supports the notion that mucins in aqueous solutions can affect superficial lipid behavior. Because addition of mucin to aqueous solutions can lower further the surface tension of the superficial lipid film and affect lipid film spread and thickness, we also endorse the notion that mucins are not present as a separate layer but instead exist throughout the entire tear film. This new concept has been supported by recent studies using laser interferometry and ultrastructural analysis. All of the above findings indicate the presence of an intimate relationship between lipids in the superficial layer and mucins in the aqueous solution.

In summary, a minute quantity of a lipid mixture was added to the surface of a saline solution that mimics the electrolyte concentration of tear fluid (0.64% NaCl, 0.0075% KCl, 0.084% CaCl.2H₂O, 0.03% MgCl₂.6H₂O, 0.39% Na acetate.3H₂O, and 0.17% Na citrate.2H₂O in each ml of water with pH adjusted to 7.4). In the absence of mucin glycoprotein, most lipid mixtures form a lipid film that is much thinner than the normal thickness noted in vivo, and granules undergoing Brownian movement. With a concentration of mucin glycoprotein in the intermediate range (preferably about 0.16 μg/ml to about 0.8 μg/ml of PMS, or its equivalent for another mucin), a uniformly thick and stable film is formed in about 20 to 40 sec. Based on this technique, the disclosed lipid composition was selected from a number of lipid mixtures that were evaluated. The disclosed formulation yields a lipid film of moderate thickness that is stable over a long period of time.

Summary of Clinical Trials

Lipid mixtures, Compositions A and B, have been used therapeutically in our Clinical Trials as described below with excellent results. See Table 2 for proportion of each lipid component in Compositions A and B. Only the purity of L-α-phosphatidylcholine used is different in the two compositions, as described above. When the formulation was tested on the eyes of human volunteers, no side effects were observed based on subjective description of symptoms, and an objective evaluation by external and biomicroscopic examinations. Furthermore, preliminary treatment of patients has confirmed the non-toxicity of the formulation and has shown encouraging effects. Within five minutes after application to the outer skin of the eyelid, close to the lid margin, many patients reported a soothing sensation, were comforted, and ocular irritation was relieved. These patients were afflicted with meibomian gland dysfunction, various forms of dry eye syndrome, abnormal lids due to chemical burns, Stevens-Johnson syndrome, ocular pemphigoid, etc.

Clinical Trials

Described below are cases in which our invented lipid ointment has shown clinical efficacy in ameliorating the patient's symptoms. Furthermore, this clinical efficacy is supported by the change of TI.

Patient No. 1

History: An 80 year old (y/o) female complained of burning sensation, dryness and ocular irritation for 15 years. These symptoms were worse just after waking up and toward the end of the day and were associated with crust in the morning in both eyes. Right eye (RE) was worse than left eye (LE). She tended to sleep on her right side. She had been seen by several ophthalmologists, who treated her unsuccessfully with artificial tears.

Examination: Visual Acuity (VA): RE: 20/30-1, LE: 20/50. Visual field was full by confrontation. Motility was full and straight. There was no afferent pupillary defect. There was no scleral show, lagophthalmos or floppy eyelid. All puncta were open and swollen. The upper and lower tear meniscus was interrupted by multiple folds of loose conjunctiva due to conjunctivochalasis, which was noted in RE worse than in LE. The conjunctiva was 1+ inflamed, RE worse than LE. Meibomian gland dysfunction was noted with meibum not expressible and meibomian gland orifice showed squamous metaplasia. Tear break up time was RE: 1 sec and LE: 2 sec. The staining was negative with rose Bengal or fluorescein. The rest of the examination was unremarkable.

Fluorescein Clearance Test (FCT): Normal aqueous tear secretion with intact reflex tearing and clearance was delayed. TI showed vertical pattern with slow spread time and thin thickness, typical of LTD. This pattern was improved immediately together with shorter spread time, and symptomatic relief when the lipid lubricant was applied to the skin along the lid margin. The patient was treated with the lipid lubricant once a day and non-preserved steroid three times a day and returned two weeks later experiencing about 80 percent improvement in burning sensation, dryness, and ocular irritation.

Patient No. 2

History: A 67 y/o female with a past ocular history of LASIK in both eyes 2.5 years ago and enhancement in LE 3 months later. Three months after LASIK she started to complain of crusty sensation and pain in the morning upon awakening and dryness all day long, RE worse than LE. She was recently diagnosed with rosacea and had 4 lids blepharoplasty 20 years ago. She has been treated with cyclosporin eye drops and non-preserved methylprednisolone without success.

Examination: Visual Acuity: RE: 20/80-2, LE: 20/20 with CL. VF was full by confrontation. Motility was full and straight. Pupils did not have APD. All puncta were swollen and open. The upper and lower tear meniscus was interrupted by multiple folds of loose conjunctiva due to conjunctivochalasis in both eyes. Pinguecula was observed on temporal bulbar conjunctiva of both eyes. Prominent conjunctivochalasis was noted in the inferior fornix of both eyes. Conjunctival injection was observed on the exposed area. Scleral show was noted 2 mm in both eyes. Meibomian gland dysfunction was evidenced in the lid margin by redness and by poor meibum expressibility.

Fluorescein Clearance Test: Normal aqueous tear secretion and delayed tear clearance (DTC). Kinetic tear interference image was also performed, which showed an LTD pattern. This pattern was immediately improved with shorter spread time when the lipid lubricant was applied to the skin of the lid margin of both upper and lower lids, her symptoms improved right away. The patient was prescribed the lipid lubricant and she returned one month later free of symptoms and vision improved 20/40 in RE.

Patient No. 3

History: A 77 y/o female complained of watery and blurry vision in RE, which occurred 30 min after waking up and lasted for the entire day. These symptoms made it more difficult for her to drive, to watch TV and to read. The symptoms tended to come and go, but became worse as the day progressed. Previously, she had been diagnosed with blepharitis and treated with autologous serum drops without success. She received cataract surgery and intraocular lens implantation successfully two years ago. She also received basal cells carcinoma removal in the right lower lid two years ago.

Examination: Visual acuity without correction was RE: 20/30-2 and LE: 20/40-1. Her blink was fine. Visual field was full by confrontation. Motility was full and straight. Pupils had no afferent papillary defect. Lids were 1+ floppy RE but not LE. There was an increase of scleral show in upgaze. All four puncta were open and swollen. Tarsus was injected. RE worse than LE. Lid margin was not inflamed. Meibomian glad dysfunction was noted with meibum poorly expressed, orifice metaplasia, and anterior migration of mucocutaneous junction. Conjunctivochalasis was noted temporally and nasally in both eyes. Pinguecula was observed on nasal and temporal bulbar conjunctiva in both eyes. One trichiasis was noted in the left lower lid. Flourescein staining was negative. TBUT was 0 sec in both eyes.

The Kinetic analysis of Tear Interference Image showed that lipid was deficient in both eyes. The image pattern, thickness and spread time of lipid and symptoms improved immediately after lipid lubricant application in lower and upper eyelid margin. To evaluate the aqueous function, the FCT was performed showing delayed tear clearance with normal aqueous secretion and reflex tearing in both eyes.

Patient No. 4

History: A fifty-seven y/o female noted in October 2002 that her eyes were dry upon waking up. She used preservative free artificial tear and contact lens to work as a teacher. Since then she started noticing more irritation with blurriness and mucus build up in LE upon awakening. She started a course of topical tobramycin eye drops with resolution. Nevertheless, contact lens intolerance continued and spread to RE. One month later she was treated with new preservative free artificial tears and FML without success. Her ophthalmologist noticed superficial punctate keratitis and dryness and suspected “lagophthalmos”. The complaints were characterized as burning and dryness without itching and pain. The symptoms were worse upon awakening. Previously, she had been taking oral tranquilizer for 30 years.

Examination: Vision was 20/20, both eyes. Her blink was fine. Visual field was full by confrontation. Motility was full and straight. Pupils had no afferent papillary defect. There were no floppy lids in both eyes. Tarsus was 1+ red. All four puncta were all open. Lid margin was not inflamed. Pinguecula were observed in the temporal exposure zone in both eyes. Meibomian gland dysfunction was noted with poor meibum expression and orifice metaplasia. Mild temporal conjunctivochalasis was noted in both eyes. Tear meniscus was low. Tear break up time were 0 sec in both eyes. The rest of examination was normal.

To evaluate the aqueous tear function, FCT was performed showing aqueous tear deficiency with reflex tearing with delayed tear clearance. We prescribed 1 percent non-preserved methyl prednisolone for two weeks. On the next visit, the patient had experienced 50 percent improvement of symptoms. We then performed on the same day Kinetic Analysis of Tear Interference Image, which showed a mixed pattern of thicker (colorful) lipids in the lower cornea and vertical striking in the upper cornea. After lipid lubricant was applied, the lipid thickness became more evenly distributed and the spread time improved as well. Additionally the patient felt better with more moisturization in both eyes. Punctal plugs were applied in both lower puncta. Three weeks later the patient felt another 50 percent better regarding dryness. TI images analyses were performed again showing a lipid layer with normal thickness, spread time and pattern.

Patient No. 5

History: A forty-eight y/o female developed Stevens Johnson syndrome with toxic epidermal necrolysis after taking Lacmital in December 2000. She complained of waking up with lids stuck together, constant eye irritation, and photophobia ever since recovery from the acute attack of Stevens Johnson syndrome. In the first six months after the illness she complained of pain and photophobia in both eyes. She tried Boston scleral lens unsuccessfully, as this resulted in rapid mucous build up and interfered with her vision, although the eyes were more comfortable. She received punctal occlusion by cautherization in all four puncta with some improvement. The use of autologous serum eye drops and lacrisert were not helpful. She used artificial tear every few minutes. Previously, she had 4 lids blepharoplasty at age of 30.

Examination: Her vision was RE: 20/20-3 and LE: 20/30+1. Visual field was full by confrontation. Motility was full and straight. Pupils had no afferent papillary defect. There was no scleral show, lagophthalmos, but lids were 1+ floppy. All four puncta were occluded, and the tear meniscus height was more than normal. Conjunctiva was quiet except for the exposed area and inferior tarsal conjunctiva, where there was injection. TBUT were 0 sec in both eyes. Lid margins showed keratinization in her temporal upper and lower eyelid of RE and nasal and temporal aspects of LE. Symblepharon were observed in the lower fornix of both eyes, RE worse than LE. MGD was noted with meibum not expressible and meibomian gland orifice showing metaplasia. Scar was found in the upper tarsus of RE and mild keratinization in the midpoint of LE. Mild trichiasis was noted.

The Kinetic Analysis of Tear Interference Images showed severe LTD, and upon application of lipid lubricant to the skin outside her eyelids, she noted immediate improvement with significant reduction of irritation and decrease in the frequency of using artificial tears. The pattern of TI also improved. She has been using the lipid lubricant daily ever since.

TABLE 4
Summary of Kinetic Analysis of Tear Interference Images

| | Before Lipid Ointment | | | After Lipid Ointment | | |
|---|---|---|---|---|---|---|
| | Spread | Thick- | | Spread | Thick- | |
| Cases | time | ness | Symptoms | time | ness | Symptoms |
| 1 | 2.1 | 40 | Dryness | 0.7 | 50 | Improved |
| 2 | 1.9 | 50 | Dryness, | 0.3 | 80 | Improved |
| | | | Pain | | | |
| 3 | 1.9 | 60 | Burning | 0.3 | 90 | Improved |
| | | | Tearing | | | |
| 4 | 0.9 | 90 | Dryness | 0.7 | 50 | Improved |
| 5 | 1.1 | 60 | Irritation | 0.5 | 80 | Improved |
| | | | Photophobia | | | |

### EQUIVALENTS

While this invention has been particularly shown and described with references to preferred embodiments thereof, it will be understood by those skilled in the art that various changes in form and details may be made therein without departing from the scope and spirit of the invention encompassed by the appended claims.

1. A composition comprising:
a) a C12 to C24 branched or unbranched hydrocarbon;
b) a mid-chain triglyceride;
c) a C26 to C36 branched or unbranched hydrocarbon;
d) a cholesteryl ester;
e) an ester of a C10 to C24 fatty acid and a C10 to C20 alcohol;
f) an ester of a C10 to C24 fatty acid and a C21 to C34 alcohol;
g) glycerol; and
h) a polar lipid.

2. The composition of claim 1, which is substantially free of water.

3. The composition of claim 1, which is substantially free of an artificial surfactant.

4. The composition of claim 1, which is in a form chosen from an ointment, a paste, and a cream.

5. The composition of claim 1, wherein the C12 to C24 hydrocarbon comprises mineral oil.

6. The composition of claim 1, wherein the mid-chain triglyceride comprises a compound of the formula CH₂(OOCR₁)CH(OOCR₂)CH₂(OOCR)₃, wherein R₁, R₂, and R₃ are the same or different and are each independently a C6 to C12 branched or unbranched alkyl group.

7. The composition of claim 1, wherein the C26 to C36 hydrocarbon comprises squalane.

8. The composition of claim 1, wherein the cholesteryl ester comprises cholesteryl behenate.

9. The composition of claim 1, wherein the ester of a C10 to C24 fatty acid and a C10 to C20 alcohol comprises steraryl palmitate or palmitic acid steraryl ester.

10. The composition of claim 1, wherein the ester of a C10 to C24 fatty acid and a C21 to C34 alcohol comprises myricyl palmitate.

11. The composition of claim 1, wherein the ester of a C10 to C24 fatty acid and a C21 to C34 alcohol comprises bleached or unbleached beeswax.

12. The composition of claim 11, wherein the beeswax is chosen from natural beeswax and artificial beeswax.

13. The composition of claim 1, wherein the polar lipid comprises a phospholipid.

14. The composition of claim 13, wherein the phospholipid comprises L-α-phosphatidylcholine.

15. The composition of claim 1, comprising:
a) from about 35 percent to about 65 percent by weight of the C12 to C24 branched or unbranched hydrocarbon;
b) from about 1 percent to about 15 percent by weight of the mid-chain triglyceride;
c) from about 10 percent to about 25 percent by weight of the C26 to C36 branched or unbranched hydrocarbon;
d) from about 5 percent to about 15 percent by weight of the cholesteryl ester;
e) from about 2 percent to about 15 percent by weight of the ester of a C10 to C24 fatty acid and a C10 to C20 alcohol;
f) from about 2 percent to about 15 percent by weight of the ester of a C10 to C24 fatty acid and a C21 to C34 alcohol;
g) from about 0.5 percent to about 5 percent by weight of the glycerol; and
h) from about 2 percent to about 10 percent by weight of the polar lipid.

16. A composition comprising:
a) mineral oil or a mixture comprising C12 to C24 alkanes;
b) a mid-chain triglyceride comprising a compound of the formula CH₂(OOCR₁)CH(OOCR₂)CH₂(OOCR)₃, wherein R₁, R₂, and R₃ are the same or different and are each independently a C6 to C12 branched or unbranched alkyl group;
c) squalane;
d) cholesteryl behenate;
e) steraryl palmitate or palmitic acid steraryl ester;
f) natural or artificial beeswax;
g) glycerol; and
h) L-α-phosphatidylcholine.

17. The composition of claim 16, comprising:
a) from about 35 percent to about 65 percent by weight of the mineral oil or the mixture comprising C12 to C24 alkanes;
b) from about 1 percent to about 15 percent by weight of the mid-chain triglyceride;
c) from about 10 percent to about 25 percent by weight of the squalane;
d) from about 5 percent to about 15 percent by weight of the cholesteryl behenate;
e) from about 2 percent to about 15 percent by weight of the steraryl palmitate or palmitic acid steraryl ester;
f) from about 2 percent to about 15 percent by weight of the ester of natural or artificial beeswax;
g) from about 0.5 percent to about 5 percent by weight of the glycerol; and
h) from about 2 percent to about 10 percent by weight of the L-α-phosphatidylcholine.

18. The composition of claim 17, wherein the mineral oil or the mixture comprising C12 to C24 alkanes is from about 40 percent to about 60 percent by weight of the composition; and the mid-chain triglyceride is from about 1 percent to about 10 percent by weight of the composition.

19. A method of making a composition for treatment of a dry eye condition in an individual in need thereof, the method comprising the steps of:
a) contacting mineral oil or a mixture comprising C12 to C24 alkanes; a mid-chain triglyceride comprising a compound of the formula CH₂(OOCR₁)CH(OOCR₂)CH₂(OOCR)₃, wherein R₁, R₂, and R₃ are the same or different and are each independently a C6 to C12 branched or unbranched alkyl group; a C26 to C36 branched or unbranched hydrocarbon; glycerol; and a polar lipid, to produce a first mixture of ingredients;
b) maintaining the first mixture at first conditions sufficient to disperse the ingredients and form a first solution or a first suspension;
c) contacting the first mixture with a cholesteryl ester; an ester of a C10 to C24 fatty acid and a C10 to C20 alcohol; an ester of a C10 to C24 fatty acid and a C21 to C34 alcohol to produce a second mixture; and
d) maintaining the second mixture at second conditions sufficient to disperse the ingredients of the first mixture with the second mixture and thereby form the composition.

20. The method of claim 19, wherein the first and second conditions comprise a temperature from about 50 degrees Celsius to about 95 degrees Celsius.

21. The method of claim 19, wherein the first conditions comprise agitating the first mixture for a first period of time sufficient to achieve homogeneity of appearance of the first mixture; and wherein the second conditions comprise agitating the second mixture for a second period of time sufficient to achieve homogeneity of appearance of the second mixture.

22. A method for treating a dry eye condition in an individual in need thereof, comprising administering a therapeutically effective amount of the composition of claim 1.

23. A method for treating a disorder chosen from lipid tear deficiency; aqueous tear deficiency; a combination of lipid tear deficiency and aqueous tear deficiency; epidermal dysplasia; Stevens Johnson Syndrome; meibomian gland diseases; rosacea; blepharitis; lagophthalmos; chemical injuries; thermal burn injuries; and diseases causing meibomian gland dysfunction, comprising administering to an individual in need thereof a therapeutically effective amount of the composition of any one of claim 16.

24. A method for treating dry eyes in an individual in need thereof, comprising:
a) using kinetic analysis of tear interference images to analyze a precorneal lipid film spread of the individual;
b) determining whether or not the precorneal lipid film spread is characteristic of lipid tear deficiency; and
if the film spread is characteristic of lipid tear deficiency, administering a therapeutically effective amount of the composition of claim 16.

25. The method of claim 23, wherein the composition is administered by a method comprising applying the composition to the outside skin of a lower eyelid or to the outside skin of an upper eyelid, and allowing the composition to diffuse onto the eye, thereby achieving sustained release of the composition and preventing or minimizing blurring of vision by the composition.

26. The method of claim 24, wherein the composition is applied to the inferior lid margin of the lower eyelid or to the superior lid margin of the upper eyelid.

27. The method of claim 22 additionally comprising administering simultaneously, separately, or sequentially; and topically to the skin, to the ocular surface, or orally, to the patient in need thereof a pharmaceutically active substance chosen from a steroid, an antibiotic, cyclosporin A, an antioxidant, and combinations thereof.

28. The method of claim 23, wherein the amount of composition that is administered in an application is from about 10 micrograms to about 50 micrograms of composition applied to an eyelid.

29. The method of claim 23, wherein the composition is applied to the outside skin of the lower eyelid and to the outside skin of the upper eyelid at least once a day for a period of time sufficient to obtain an improvement in the dry eye condition or a decrease in severity of a symptom of the dry eye condition.

30. The method of claim 25, wherein the composition is applied to about one square centimeter of eyelid surface in each administration.

31. The method of claim 29, wherein the composition is applied from about 1 time per day to about 6 times per day.

32. The method of claim 23, wherein the composition is applied by urging the composition out of at least one restricted discharge aperture in a frontal edge of an applicator comprising:
a hollow housing defining a cylindrical-shaped reservoir containing the composition, the reservoir in fluid communication with the aperture;
a plunger for resiliently urging the composition out of the reservoir through the aperture, the plunger adapted to fit within the reservoir and to be moveable into the reservoir; and
an actuator means for selectively moving the plunger from a first position wherein the composition is within the reservoir to a second position wherein a portion of the composition is urged through the aperture and applied to the outside skin of the lower eyelid or to the outside skin of the upper eyelid.

33. (canceled)

34. A method for treating a dry eye condition by administering an ointment comprising at least one lipid to an individual in need thereof, while achieving sustained release of the ointment and preventing a blurring of vision by the ointment, the method comprising administering a therapeutically effective amount of the ointment to the inferior lid margin of the outside skin of the lower eyelid or to the superior lid margin of the outside skin of the upper eyelid, and allowing the ointment to diffuse onto the eye.

35. The method of claim 34, wherein the ointment is substantially free of water and substantially free of an artificial surfactant.

36.-38. (canceled)

39. The composition of claim 16, wherein the composition has the following properties that can be observed when the composition is placed on a lid margin and allowed to diffuse over an ocular surface of an individual in need thereof:
a) capable of improving a pre-corneal lipid film spread characterized by vertical tear interference pattern; and
b) capable of decreasing tear spread time in an individual by a factor of from about 0.2 to about 0.8 as determined by a kinetic analysis of tear film spread.
